(19) United States
(12) Patent Application Publication
(10) Pub. No.: US 2023/0088878 A1
(43) Pub. Date: Mar. 23, 2023

(54) HAIR CARE FORMULATION

(71) Applicants: Rohm and Haas Company, Collegeville, PA (US); Union Carbide Corporation, Seadrift, TX (US)

(72) Inventors: Nikhil J. Fernandes, Philadelphia, PA (US); Emmett M. Partain, III, Bound Brook, NJ (US); Lyndsay M. Leal, Spring City, PA (US); Jennifer P. Todd, Willow Grove, PA (US); Michaeleen Pacholski, Collegeville, PA (US)

(21) Appl. No.: 17/802,562

(22) PCT Filed: Mar. 17, 2021
(86) PCT No.: PCT/US2021/022649

§ 371 Date: Aug. 26, 2022

Related U.S. Application Data
(60) Provisional application No. 62/993,779, filed on Mar. 24, 2020.

(51) Int. Cl. A61K 8/73; A61K 8/891; A61Q 5/10; A61Q 5/02

(52) U.S. Cl. CPC A61K 8/73; A61K 8/891; A61Q 5/10; A61Q 5/02; A61K 2800/5426; A61K 2800/48

(57) ABSTRACT

A hair care formulation is provided, comprising: a dermatologically acceptable vehicle; a dermatologically acceptable silicone; and a deposition aid polymer, wherein the deposition aid polymer is a branched chain cationic dextran polymer, comprising a dextran polymer functionalized with quaternary ammonium groups; wherein the dextran polymer has a weight average molecular weight of 100,000 to 650,000 Daltons; and wherein the deposition aid polymer enhances deposition of silicone from the hair care formulation onto mammalian hair.

The present invention relates to a hair care formulation. In particular, the present invention relates to a hair care formulation containing: a dermatologically acceptable vehicle; a dermatologically acceptable silicone; and a deposition aid polymer, wherein the deposition aid polymer is a cationic dextran polymer, comprising a dextran polymer functionalized with quaternary ammonium groups; wherein the dextran polymer has a weight average molecular weight of 100,000 to 650,000 Daltons; and wherein the deposition aid polymer enhances deposition of silicone from the hair care formulation onto mammalian hair.

Deposition of silicone is of particular interest for various personal care compositions. In particular, there is interest for personal care cleansers (e.g., body wash, face wash, hand wash, soap, shampoo, shampoo conditioners and hair conditioners) that provide moisturizing/conditioning benefits in addition to cleaning benefits.

Hair cleansing has become an ubiquitous component of personal hygiene. Cleansing of the hair facilitates the removal of dirt, germs and other things that are perceived as harmful to the hair or the individual. Cleansing formulations typically including a surfactant to promote the removal of materials deposited on the hair. Unfortunately, the cleansing formulations remove both undesirable and desirable materials from hair. For example, cleansing formulations frequently undesirably remove oils from hair, which oils operate to protect hair from loss of moisture. Removal of too much oil from hair may leave the hair vulnerable to becoming dry and damaged. One solution to this concern is the selection of mild surfactants. Another approach is to incorporate additives that help replace the oils removed through deposition; however, this approach has proven difficult in implementation, particularly in rinse off applications.

In U.S. Pat. No. 7,067,499, Erazo-Majewicz, et al disclose personal care and household care product composition comprising at least one cationic polygalactomannan or a derivative of cationic polygalactomannans wherein the derivative moiety on the cationic derivatized polygalactomannan is selected from the group consisting of alkyl, hydroxyalkyl, alkylhydroxyalkyl, and carboxymethyl wherein the alkyl has a carbon chain containing from 1 to 22 carbons and the hydroxyalkyl is selected from the group consisting of hydroxyethyl, hydroxypropyl, and hydroxybutyl, wherein the at least one cationic polygalactomannan or derivative of cationic polygalactomannans have a mean average molecular weight (Mw) having a lower limit of 5,000 and an upper limit of 200,000 and having a light transmittance in a 10% aqueous solution of greater than 80% at a light wavelength of 600 nm and a protein content of less than 1.0% by weight of polysaccharide, and aldehyde functionality content of at least 0.01 meg/gram.

While conventionally used deposition aids such as soluble cationic modified celluloses (e.g., polyquaternium-10), guar hydroxypropyltrimonium chloride and other cationic polymers (e.g., polyquaternium-6, polyquaternium-7) provide a certain level of deposition in personal care cleansers; they nevertheless exhibit low efficiency necessitating a relatively high incorporation of the active into the personal care cleanser formulation to facilitate desired results. Such high active (e.g., silicone) levels, however, detrimentally effect the foam/lathery in use consumer feel of the formulation and cost.

Accordingly, there remains a need for deposition aids that facilitate enhanced efficiency of silicone deposition from hair care formulations.

The present invention provides a hair care formulation, comprising: a dermatologically acceptable vehicle; a dermatologically acceptable silicone; and a deposition aid polymer, wherein the deposition aid polymer is a cationic dextran polymer, comprising a dextran polymer functionalized with quaternary ammonium groups; wherein the dextran polymer has a weight average molecular weight of 100,000 to 650,000 Daltons; and wherein the deposition aid polymer enhances deposition of silicone from the cleansing formulation onto mammalian hair.

The present invention provides a method of depositing silicone on to mammalian hair, comprising: selecting a hair care formulation of the present invention; and applying the hair care formulation to mammalian hair.

### DETAILED DESCRIPTION

We have surprisingly found that silicone deposition from hair care formulations can be enhanced through incorporation of a deposition aid polymer, wherein the deposition aid polymer is a cationic dextran polymer, comprising a dextran polymer functionalized with quaternary ammonium groups; wherein the dextran polymer has a weight average molecular weight of 100,000 to 650,000 Daltons; and wherein the deposition aid polymer enhances deposition of silicone from the cleansing formulation onto mammalian hair.

Unless otherwise indicated, ratios, percentages, parts, and the like are by weight.

As used herein, unless otherwise indicated, the phrase “molecular weight” or M_{w} refers to the weight average molecular weight as measured in a conventional manner with gel permeation chromatography (GPC) and conventional standards, such as polyethylene glycol standards. GPC techniques are discussed in detail in Modern Size Exclusion Chromatography, W. W. Yau, J. J. Kirkland, D. D. Bly; Wiley-Interscience, 1979, and in A Guide to Materials Characterization and Chemical Analysis, J. P. Sibilia; VCH, 1988, p. 81-84. Molecular weights are reported herein in units of Daltons, or equivalently, g/mol.

The term “dermatologically acceptable” as used herein and in the appended refers to ingredients that are typically used for topical application to the skin, and is intended to underscore that materials that are toxic when present in the amounts typically found in skin care compositions are not contemplated as part of the present invention.

Preferably, the hair care formulation of the present invention is selected from the group consisting of a shampoo, a conditioning shampoo, a leave on hair conditioner, a rinse off hair conditioner, a hair coloring agent and a hair styling gel. More preferably, the hair care formulation of the present invention is selected from the group consisting of a shampoo, a conditioning shampoo, a leave on hair conditioner and a rinse off hair conditioner. Still more preferably, the hair care formulation of the present invention is a shampoo, a conditioner or a conditioning shampoo. Most preferably, the hair care formulation of the present invention is a shampoo or a conditioning shampoo.

Preferably, the hair care formulation of the present invention, comprises: a dermatologically acceptable vehicle (preferably, wherein the hair care formulation comprises 25 to 99 wt % (preferably, 30 to 95 wt %; more preferably, 40 to 90 wt %; most preferably, 70 to 85 wt %), based on weight of the hair care formulation, of a dermatologically acceptable vehicle); a dermatologically acceptable silicone (preferably, wherein the hair care formulation comprises 0.1 to 5 wt % (more preferably, 0.15 to 4 wt %; still more preferably, 0.25 to 2 wt %; most preferably, 0.4 to 1.5 wt %), based on weight of the hair care formulation, of the dermatologically acceptable silicone; and a deposition aid polymer (preferably, wherein the hair care formulation comprises 0.1 to 1 wt % (preferably, 0.15 to 0.75 wt %; more preferably, 0.2 to 0.5 wt %; most preferably, 0.25 to 0.4 wt %), based on weight of the hair care formulation, of the deposition aid polymer), wherein the deposition aid polymer is a cationic dextran polymer, comprising a dextran polymer functionalized with quaternary ammonium groups; wherein the dextran polymer has a weight average molecular weight of 100,000 to 650,000 Daltons (preferably, 125,000 to 600,000 Daltons; more preferably, 130,000 to 575,000 Daltons; most preferably, 145,000 to 525,000 Daltons); and wherein the deposition aid polymer enhances deposition of silicone from the cleansing formulation onto mammalian hair.

Preferably, the hair care formulation of the present invention is a liquid formulation. More preferably, the hair care formulation of the present invention is an aqueous liquid formulation.

Preferably, the hair care formulation of the present invention, comprises a dermatologically acceptable vehicle. More preferably, the hair care formulation of the present invention, comprises: 25 to 99 wt % (preferably, 30 to 97.5 wt %; more preferably, 60 to 95 wt %; most preferably, 75 to 90 wt %), based on weight of the hair care formulation, of a dermatologically acceptable vehicle. Still more preferably, the hair care formulation of the present invention, comprises: 25 to 99 wt % (preferably, 30 to 97.5 wt %; more preferably, 60 to 95 wt %; most preferably, 75 to 90 wt %), based on weight of the hair care formulation, of a dermatologically acceptable vehicle; wherein the dermatologically acceptable vehicle comprises water. Yet more preferably, the hair care formulation of the present invention, comprises: 25 to 99 wt % (preferably, 30 to 97.5 wt %; more preferably, 60 to 95 wt %; most preferably, 75 to 90 wt %), based on weight of the hair care formulation, of a dermatologically acceptable vehicle; wherein the dermatologically acceptable vehicle is selected from the group consisting of water and an aqueous C₁-4 alcohol mixture. Most preferably, the hair care formulation of the present invention, comprises: 25 to 99 wt % (preferably, 30 to 97.5 wt %; more preferably, 60 to 95 wt %; most preferably, 75 to 90 wt %), based on weight of the hair care formulation, of a dermatologically acceptable vehicle; wherein the dermatologically acceptable vehicle is water.

Preferably, the water used in the hair care formulation of the present invention is at least one of distilled water and deionized water. More preferably, the water used in the hair care formulation of the present invention is distilled and deionized.

Preferably, the hair care formulation of the present invention comprises a dermatologically acceptable silicone. More preferably, the hair care formulation of the present invention comprises 0.1 to 5 wt % (preferably, 0.15 to 4 wt %; more preferably, 0.25 to 2 wt %; most preferably, 0.4 to 1.5 wt %), based on weight of the hair care formulation, of a dermatologically acceptable silicone (preferably, wherein the dermatologically acceptable silicone conditions hair). Still more preferably, the hair care formulation of the present invention comprises 0.1 to 5 wt % (preferably, 0.15 to 4 wt %; more preferably, 0.25 to 2 wt %; most preferably, 0.4 to 1.5 wt %), based on weight of the hair care formulation, of a dermatologically acceptable silicone, wherein the dermatologically acceptable silicone is selected from the group consisting of amodimethicone, cyclomethicone, dimethicone, dimethiconol, hexadecyl methicone, hexamethyldisiloxane, methicone, phenyl dimethicone, stearoxy dimethicone polyalkyl siloxane, polyalkylaryl siloxane, silicone gums (i.e., polydiorganosiloxanes having a weight average molecular weight of 200,000 to 1,000,000 Daltons), polyaminofunctional silicones (e.g., Dow Corning® 929) and mixtures thereof. Yet more preferably, the hair care formulation of the present invention comprises 0.1 to 5 wt % (preferably, 0.15 to 4 wt %; more preferably, 0.25 to 2 wt %; most preferably, 0.4 to 1.5 wt %), based on weight of the hair care formulation, of a dermatologically acceptable silicone, wherein the dermatologically acceptable silicone is selected from the group consisting of amodimethicone, cyclomethicone, dimethicone, dimethiconol, hexadecyl methicone, hexamethyldisiloxane, methicone, phenyl dimethicone, stearoxy dimethicone and mixtures thereof. Still yet more preferably, the hair care formulation of the present invention comprises 0.1 to 5 wt % (preferably, 0.15 to 4 wt %; more preferably, 0.25 to 2 wt %; most preferably, 0.4 to 1.5 wt %), based on weight of the hair care formulation, of a dermatologically acceptable silicone, wherein the dermatologically acceptable silicone is selected from the group consisting of amodimethicone, cyclomethicone, dimethicone, dimethiconol, hexadecyl methicone, methicone and mixtures thereof. Yet still more preferably, the hair care formulation of the present invention comprises 0.1 to 5 wt % (preferably, 0.15 to 4 wt %; more preferably, 0.25 to 2 wt %; most preferably, 0.4 to 1.5 wt %), based on weight of the hair care formulation, of a dermatologically acceptable silicone, wherein the dermatologically acceptable silicone is selected from the group consisting of amodimethicone, dimethicone, dimethiconol and a mixture thereof. Most preferably, the hair care formulation of the present invention comprises 0.1 to 5 wt % (preferably, 0.15 to 4 wt %; more preferably, 0.25 to 2 wt %; most preferably, 0.4 to 1.5 wt %), based on weight of the hair care formulation, of a dermatologically acceptable silicone, wherein the dermatologically acceptable silicone comprises a dimethiconol.

Preferably, the hair care formulation of the present invention comprises a deposition aid polymer, wherein the deposition aid polymer is a cationic dextran polymer, comprising a dextran polymer functionalized with quaternary ammonium groups; wherein the dextran polymer has a weight average molecular weight of 100,000 to 650,000 (preferably, 125,000 to 600,000; more preferably, 130,000 to 575,000; most preferably, 145,000 to 525,000) Daltons; and wherein the deposition aid polymer enhances deposition of silicone from the hair care formulation onto mammalian hair. More preferably, the hair care formulation of the present invention comprises 0.1 to 1 wt % (preferably, 0.15 to 0.75 wt %; more preferably, 0.2 to 0.5 wt %; most preferably, 0.25 to 0.4 wt %), based on weight of the hair care formulation, of a deposition aid polymer, wherein the deposition aid polymer is a cationic dextran polymer, comprising a dextran polymer functionalized with quaternary ammonium groups; wherein the dextran polymer has a weight average molecular weight of 100,000 to 650,000 (preferably, 125,000 to 600,000; more preferably, 130,000 to 575,000; most preferably, 145,000 to 525,000) Daltons; and wherein the deposition aid polymer enhances deposition of silicone from the hair care formulation onto mammalian hair. Most preferably, the hair care formulation of the present invention comprises 0.1 to 1 wt % (preferably, 0.15 to 0.75 wt %; more preferably, 0.2 to 0.5 wt %; most preferably, 0.25 to 0.4 wt %), based on weight of the hair care formulation, of a deposition aid polymer; wherein the deposition aid polymer is a cationic dextran polymer, comprising a dextran polymer functionalized with quaternary ammonium groups; wherein the dextran polymer has a weight average molecular weight of 100,000 to 650,000 (preferably, 125,000 to 600,000; more preferably, 130,000 to 575,000; most preferably, 145,000 to 525,000) Daltons; wherein the cationic dextran polymer has a Kjeldahl nitrogen content corrected for ash and volatiles, TKN, of 0.5 to 4.0 wt % (preferably, 0.75 to 3.25 wt %; more preferably, 0.9 to 2.6 wt %; most preferably, 1.0 to 2.0 wt %)(measured using a Buchi KjelMaster K-375 automated analyzer, corrected for volatiles and ash measured as described in ASTM method D-2364); and wherein the deposition aid polymer enhances deposition of silicone from the hair care formulation onto mammalian hair.

Preferably, the dextran polymer is a branched chain dextran polymer. More preferably, the dextran polymer is a branched chain dextran polymer; wherein the branched chain dextran polymer comprises a plurality of glucose structural units; wherein 90 to 98 mol % (preferably, 92.5 to 97.5 mol %; more preferably, 93 to 97 mol %; most preferably, 94 to 96 mol %) of the glucose structural units are connected by α-D-1,6 linkages and 2 to 10 mol % (preferably, 2.5 to 7.5 mol %; more preferably, 3 to 7 mol %; most preferably, 4 to 6 mol %) of the glucose structural units are connected by α-1,3 linkages. Most preferably, the dextran polymer is a branched chain dextran polymer; wherein the branched chain dextran polymer comprises a plurality of glucose structural units; wherein 90 to 98 mol % (preferably, 92.5 to 97.5 mol %; more preferably, 93 to 97 mol %; most preferably, 94 to 96 mol %) of the glucose structural units are connected by α-D-1,6 linkages and 2 to 10 mol % (preferably, 2.5 to 7.5 mol %; more preferably, 3 to 7 mol %; most preferably, 4 to 6 mol %) of the glucose structural units are connected by α-1,3 linkages according to formula I

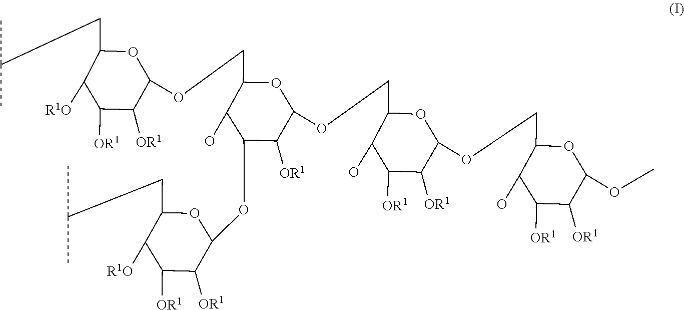

wherein R¹ is selected from a hydrogen, a C₁₋₄ alkyl group and a hydroxy C₁₋₄ alkyl group; and wherein the average branch off the dextran polymer backbone is ≤3 anhydroglucose units.

Preferably, the dextran polymer contain less than 0.01 wt %, based on weight of the dextran polymer, of alternan. More preferably, the dextran polymer contain less than 0.001 wt %, based on weight of the dextran polymer, of alternan. Most preferably, the dextran polymer contain less than the detectable limit of alternan.

Preferably, the deposition aid polymer is a cationic dextran polymer; wherein the cationic dextran polymer, comprises a dextran polymer functionalized with quaternary ammonium groups. More preferably, the deposition aid polymer is a cationic dextran polymer; wherein the cationic dextran polymer, comprises a dextran polymer functionalized with quaternary ammonium groups; wherein the quaternary ammonium groups are selected from the group consisting of quaternary ammonium moieties of formula (A) bound to a pendent oxygen on the dextran polymer

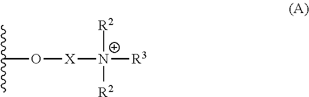

wherein

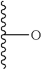 [SMILES: *O]

is a pendant oxygen on the dextran polymer; wherein X is a divalent linking group bonding the quaternary ammonium moiety to the pendent oxygen on the dextran polymer (preferably, wherein X is selected from divalent hydrocarbon groups, which may optionally be substituted (e.g., with a hydroxy group, an alkoxy group, an ether group); more preferably, wherein X is a —CH₂CH(OR⁴)CH₂— group; wherein R⁴ is selected from the group consisting of a hydrogen and a C₁₋₄ alkyl group (preferably, a hydrogen); most preferably, X is a —CH₂CH(OH)CH₂— group); wherein each R² is independently selected from the group consisting of a C₁₋₇ alkyl group (preferably, a C₁₋₃ alkyl group; more preferably, a methyl group and an ethyl group; most preferably, a methyl group); and wherein R³ is selected from the group consisting of a C₁₋₂₂ alkyl group (preferably, selected from the group consisting of a C₁₋₃ alkyl group and a C₆₋₂₂ alkyl group; more preferably, a methyl group and an ethyl group; most preferably, a methyl group). Most preferably, the deposition aid polymer is a cationic dextran polymer; wherein the cationic dextran polymer, comprises a dextran polymer functionalized with quaternary ammonium groups; wherein the quaternary ammonium groups are selected from the group consisting of quaternary ammonium moieties of formula (B) bound to a pendent oxygen on the dextran polymer

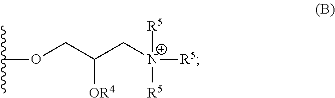

wherein R⁴ is selected from the group consisting of a hydrogen and a C₁₋₄ alkyl group (preferably, a hydrogen); and wherein each R⁵ is independently selected from the group consisting of a methyl group and an ethyl group (preferably, a methyl group).

Preferably, the deposition aid polymer comprises <0.001 meg/gram (preferably, <0.0001 meq/gram; more preferably, <0.00001 meq/gram; most preferably, <detectable limit) of aldehyde functionality.

Preferably, the deposition aid polymer comprises <0.1% (preferably, <0.01%; more preferably, <0.001%; most preferably, <detectable limit), of the linkages between individual glucose units in the deposition aid polymer are β-1,4 linkages.

Preferably, the deposition aid polymer comprises <0.1% (preferably, <0.01%; more preferably, <0.001%; most preferably, <detectable limit), of the linkages between individual glucose units in the deposition aid polymer are β-1,3 linkages.

Preferably, the deposition aid polymer comprises <0.001 meq/gram (preferably, <0.0001 meq/gram; more preferably, <0.00001 meq/gram; most preferably, <detectable limit) of silicone containing functionality.

Preferably, the hair care formulation of the present invention, optionally, further comprises at least one additional ingredient selected from the group consisting of a hair care cleansing surfactant; an antimicrobial agent/preservative (e.g., benzoic acid, sorbic acid, phenoxyethanol, methylisothiazolinone); a rheology modifier (e.g., PEG-150 pentaerythrityl tetrastearate); a soap; a colorant; pH adjusting agent; an antioxidant (e.g., butylated hydroxytoluene); a humectant (e.g., glycerin, sorbitol, monoglycerides, lecithins, glycolipids, fatty alcohols, fatty acids, polysaccharides, sorbitan esters, polysorbates (e.g., Polysorbate 20, Polysorbate 40, Polysorbate 60, and Polysorbate 80), diols (e.g., propylene glycol), diol analogs, triols, triol analogs, cationic polymeric polyols); a wax; a foaming agent; an emulsifying agent; a colorant; a fragrance; a chelating agent (e.g., tetrasodium ethylene diamine tetraacetic acid); a preservative (e.g., benzoic acid, sorbic acid, phenoxyethanol, methylisothiazolinone); a bleaching agent; a lubricating agent; a sensory modifier; a sunscreen additive; a vitamin; a protein/amino acid; a plant extract; a natural ingredient; a bioactive agent; an anti-aging agent; a pigment; an acid; a penetrant; an anti-static agent; an anti-frizz agent; an antidandruff agent; a hair waving/straightening agent; a hair styling agent; a hair oil; an absorbent; a hard particle; a soft particle; a conditioning agent (e.g., guar hydroxypropyltrimonium chloride, PQ-10, PQ-7); a slip agent; an opacifier; a pearlizing agent and a salt. More preferably, the hair care formulation of the present invention, optionally, further comprises at least one additional ingredient selected from the group consisting of a hair care cleansing surfactant; an antimicrobial agent/preservative (e.g., benzoic acid, sorbic acid, phenoxyethanol, methylisothiazolinone); a rheology modifier (e.g., PEG-150 pentaerythrityl tetrastearate); and a chelating agent (e.g., tetrasodium ethylene diamine tetraacetic acid). Most preferably, the hair care formulation of the present invention, optionally, further comprises at least one additional ingredient selected from the group consisting of a hair care cleansing surfactant; an antimicrobial agent/preservative mixture of phenoxyethanol and methylisothiazolinone; PEG-150 pentaerythrityl tetrastearate; tetrasodium ethylene diamine tetraacetic acid and a mixture of phenoxyethanol and methylisothiazolinone.

Preferably, the hair care formulation of the present invention further comprises a hair care cleaning surfactant. More preferably, the hair care formulation of the present invention further comprises a hair care cleansing surfactant, wherein the hair care cleansing surfactant is selected from the group consisting of alkyl polyglucosides (e.g., lauryl glucoside, coco-glucoside, decyl glucoside), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfates (e.g., sodium lauryl ether sulfate (SLES)), sulfonates (e.g., sodium C₁₄₋₁₆ olefin sulfonate), succinates (e.g., disodium lauryl sulfosuccinate), fatty alkanolamides (e.g., cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine) and mixtures thereof. Still more preferably, the hair care formulation of the present invention further comprises a hair care cleaning surfactant; wherein the hair care formulation is selected from the group consisting of a shampoo and a conditioning shampoo; and wherein the hair care cleaning surfactant is selected from the group consisting of alkyl polyglucosides (e.g., lauryl glucoside, coco-glucoside, decyl glucoside), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfates (e.g., sodium lauryl ether sulfate (SLES)), sulfonates (e.g., sodium C₁₄₋₁₆ olefin sulfonate), succinates (e.g., disodium lauryl sulfosuccinate), fatty alkanolamides (e.g., cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine) and mixtures thereof. Most preferably, the hair care formulation of the present invention further comprises a hair care cleaning surfactant; wherein the hair care formulation is selected from the group consisting of a shampoo and a conditioning shampoo; and wherein the hair care cleaning surfactant comprises a mixture of a betaine (preferably, cocamidopropyl betaine), a sulfate (preferably, sodium lauryl ether sulfate (SLES)), and a fatty alkanolamide (preferably, cocamide monoethanolamine).

Preferably, the hair care formulation of the present invention further comprises 0.01 to 80 wt % (more preferably, 1 to 50 wt %; still more preferably, 5 to 20 wt %, most preferably, 7 to 15 wt %), based on weight of the hair care formulation, of a hair care cleaning surfactant. More preferably, the hair care formulation of the present invention further comprises 0.01 to 80 wt % (more preferably, 1 to 50 wt %; still more preferably, 5 to 20 wt %, most preferably, 7 to 15 wt %), based on weight of the hair care formulation, of a hair care cleaning surfactant, wherein the hair care cleaning surfactant is selected from the group consisting of alkyl polyglucosides (e.g., lauryl glucoside, coco-glucoside, decyl glucoside), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfates (e.g., sodium lauryl ether sulfate (SLES)), sulfonates (e.g., sodium C₁₄₋₁₆ olefin sulfonate), succinates (e.g., disodium lauryl sulfosuccinate), fatty alkanolamides (e.g., cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine) and mixtures thereof. Still more preferably, the hair care formulation of the present invention further comprises 0.01 to 80 wt % (more preferably, 1 to 50 wt %; still more preferably, 5 to 20 wt %, most preferably, 7 to 15 wt %), based on weight of the hair care formulation, of a hair care cleaning surfactant; wherein the hair care formulation is a body wash formulation and wherein the hair care cleaning surfactant is selected from the group consisting of alkyl polyglucosides (e.g., lauryl glucoside, coco-glucoside, decyl glucoside), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfates (e.g., sodium lauryl ether sulfate (SLES)), sulfonates (e.g., sodium C₁₄₋₁₆ olefin sulfonate), succinates (e.g., disodium lauryl sulfosuccinate), fatty alkanolamides (e.g., cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine) and mixtures thereof. Most preferably, the hair care formulation of the present invention further comprises 0.01 to 80 wt % (more preferably, 1 to 50 wt %; still more preferably, 5 to 20 wt %, most preferably, 7 to 15 wt %), based on weight of the hair care formulation, of a hair care cleaning surfactant; wherein the hair care formulation is a body wash formulation and wherein the hair care cleaning surfactant comprises a mixture of a betaine (preferably, cocamidopropyl betaine), a sulfate (preferably, sodium lauryl ether sulfate (SLES)), and a fatty alkanolamide (preferably, cocamide monoethanolamine).

Preferably, the hair care formulation further comprises a thickener. More preferably, the hair care formulation further comprises a thickener, wherein the thickener is selected to increase the viscosity of the hair care formulation, preferably without substantially modifying the other properties of the hair care formulation. Preferably, the hair care formulation further comprises a thickener, wherein the thickener is selected to increase the viscosity of the hair care formulation, preferably without substantially modifying the other properties of the hair care formulation and wherein the thickener accounts for 0 to 5.0 wt % (preferably, 0.1 to 5.0 wt %; more preferably, 0.2 to 2.5 wt %; most preferably, 0.5 to 2.0 wt %), based on weight of the hair care formulation.

Preferably, the hair care formulation of the present invention further comprises an antimicrobial agent/preservative. More preferably, the hair care formulation of the present invention further comprises an antimicrobial/preservative, wherein the antimicrobial/preservative is selected from the group consisting of phenoxyethanol, benzoic acid, benzyl alcohol, sodium benzoate, DMDM hydantoin, 2-ethylhexyl glyceryl ether, isothiazolinone (e.g., methylchloroisothiazolinone, methylisothiazolinone) and mixtures thereof. Still more preferably, the hair care formulation of the present invention, further comprises an antimicrobial/preservative, wherein the antimicrobial/preservative is a mixture of phenoxyethanol and an isothiazolinone (more preferably, wherein the antimicrobial/preservative is a mixture of phenoxyethanol and methylisothiazolinone).

Preferably, the hair care formulation of the present invention optionally further comprises a pH adjusting agent. More preferably, the hair care formulation of the present invention, further comprises a pH adjusting agent, wherein the hair care formulation has a pH of 4 to 9 (preferably, 4.25 to 8; more preferably, 4.5 to 7; most preferably, 4.75 to 6).

Preferably, the pH adjusting agent is selected from the group consisting of at least one of citric acid, lactic acid, hydrochloric acid, aminoethyl propanediol, triethanolamine, monoethanolamine, sodium hydroxide, potassium hydroxide, amino-2-methyl-1-propanol. More preferably, the pH adjusting agent is selected from the group consisting of at least one of citric acid, lactic acid, sodium hydroxide, potassium hydroxide, triethanolamine, amino-2-methyl-1-propanol. Still more preferably, the pH adjusting agent includes citric acid. Most preferably, the pH adjusting agent is citric acid.

Preferably, the method of depositing silicone on to mammalian hair of the present invention, comprises: selecting a hair care formulation of the present invention and applying the hair care formulation to mammalian hair. More preferably, the method of depositing silicone on to mammalian hair of the present invention, further comprises: rinsing the hair care formulation from the mammalian hair with a rinse water. Most preferably, the method of depositing silicone on to mammalian hair of the present invention, comprises: selecting a hair care formulation of the present invention; applying the hair care formulation to mammalian hair; and rinsing the hair care formulation from the mammalian hair; wherein the hair care formulation is at least one of a shampoo and a conditioner (preferably, wherein the at least 10 mol % (more preferably, at least 12 mol %; most preferably, at least 15 mol %) of the silicone from the composition is deposited on the mammalian hair).

Some embodiments of the present invention will now be described in detail in the following Examples.

### Example S1: Synthesis of Cationic Dextran Polymer

A 500 mL, four necked, round bottom flask fitted with a rubber serum cap, a nitrogen inlet, a pressure equalizing addition funnel, a stirring paddle and motor, a subsurface thermocouple connected to a J-KEM controller and a Friedrich condenser connected to a mineral oil bubbler was charged with dextran polymer (30.33 g; Sigma-Aldrich product D4876) and deionized water (160.75 g). The weight average molecular weight of the dextran polymer was 100,000 to 200,000 Daltons. The addition funnel was charged with a 70% aqueous solution of 2,3-epoxypropyltrimethylammonium chloride (27.13 g; QUAB® 151 available from SKW QUAB Chemicals). The flask contents were allowed to stir until the dextran polymer dissolved in the deionized water. While the contents were stirring, the apparatus was purged with nitrogen to displace any oxygen entrained in the system. The nitrogen flow rate was about 1 bubble per second. The mixture was purged with nitrogen while stirring for one hour. Using a plastic syringe, a 25% aqueous sodium hydroxide solution (4.76 g) was added over a period of a few minutes to the flask contents with stirring under nitrogen. The flask contents were then allowed to stir under nitrogen for 30 minutes. The contents of the addition funnel were then charged to the flask contents dropwise over a few minutes under nitrogen with continued stirring. After the contents of the addition funnel were transferred to the flask contents, the mixture was allowed to stir for 5 minutes. Then heat was applied to the flask contents with a heating mantle controlled using the J-KEM controller set at 55° C. The flask contents were heated to and maintained at 55° C. for 90 minutes. The flask contents were then cooled to room temperature while maintaining a positive nitrogen pressure in the flask. When the flask contents reached room temperature, glacial acetic acid (2.5 g) was added. The polymer was recovered by non-solvent precipitation from methanol, recovering the precipitated polymer by vacuum filtration using a Buchner funnel and dried overnight in vacuo at 50° C. The product branched chain cationic dextran polymer was an off-white solid (24.3 g), with a volatiles content of 3.65%, an ash content of 0.37% (as sodium chloride). The volatiles and ash were measured as described in ASTM method D-2364. The Kjeldahl nitrogen content, TKN, was measured using a Buchi KjelMaster K-375 automated analyzer, and was found to be 1.41% (corrected for volatiles and ash), which corresponds to a trimethylammonium degree of substitution of 0.19.

### Examples S2-S4: Synthesis of Cationic Dextran Polymer

Cationic dextran polymers were prepared substantially as described in Example S1 but varying the dextran starting material as noted in TABLE 1.

TABLE 1

| | | Linkages | | Kjeldahl | Molecular |
|---|---|---|---|---|---|
| Example | Dextran | α-D-(1-6) | α (1-3) | Nitrogen (wt %) | weight (Da) |
| S1 | D4876^{a} | 95% | 5% | 1.41 | ~150,000 |
| S2 | D1662^{a} | 95% | 5% | 1.30 | ~40,000 |
| S3 | D5376^{a} | 95% | 5% | 1.19 | ~2,000,000 |
| S4 | Dextran | — | — | 1.32 | ~500,000 |
| | powder^{b} | | | | |

^{a}available from Sigma-Aldrich
^{b}available from Dextran Products Limited

### Comparative Examples CF1-CF4 and Examples F1-F7: Shampoo Formulations

A shampoo formulation was prepared in each of Comparative Examples CF1-CF4 and Examples F1-F7 using the generic shampoo formulation noted in TABLES 2 and 3.

TABLE 2

| | CF1 | CF2 | CF3 | F1 | F2 | F3 | F4 |
|---|---|---|---|---|---|---|---|
| Ingredient INCI name | wt % | wt % | wt % | wt % | wt % | wt % | wt % |
| Deionized water | q.s. 100 | q.s. 100 | q.s. 100 | q.s. 100 | q.s. 100 | q.s. 100 | q.s. 100 |
| 30 wt % aq. soln. Sodium Lauryl Ether Sulfate¹ | 30 | 30 | 30 | 30 | 30 | 30 | 30 |
| Polyquaternium-10² | 0.3 | — | — | — | — | — | — |
| Guar hydroxypropyltrimonium chloride³ | — | — | 0.3 | — | — | — | — |
| Unmodified dextran⁴ | — | 0.3 | — | — | — | — | — |
| Example S1 | — | — | — | 0.3 | — | — | — |
| Example S2 | — | — | — | — | 0.3 | — | — |
| Example S3 | — | — | — | — | — | 0.3 | — |
| Example S4 | — | — | — | — | — | — | 0.3 |
| Tetrasodium EDTA⁵ | 0.2 | 0.2 | 0.2 | 0.2 | 0.2 | 0.2 | 0.2 |
| 45 wt % aq. soln. PEG-150 Pentaerythrityl | 1.0 | 1.0 | 1.0 | 1.0 | 1.0 | 1.0 | 1.0 |
| Tetrastearate⁶ | | | | | | | |
| Cocamide MEA⁷ | 1.0 | 1.0 | 1.0 | 1.0 | 1.0 | 1.0 | 1.0 |
| 30 wt % aq. soln. Cocamidopropyl Betaine⁸ | 6.0 | 6.0 | 6.0 | 6.0 | 6.0 | 6.0 | 6.0 |
| Phenoxyethanol and Methyliasothiazolinone⁹ | 0.5 | 0.5 | 0.5 | 0.5 | 0.5 | 0.5 | 0.5 |
| 50 wt % aq. soln. Dimethiconol and | 2.0 | 2.0 | 2.0 | 2.0 | 2.0 | 2.0 | 2.0 |
| TEA-dodecylbenzenesulfonate¹⁰ | | | | | | | |

¹available from Stepan Company under tradename Steol ® CS-130
²available from The Dow Chemical Company under tradename UCARE ™ LR-400
³available from Solvay Novecare under tradename Jaguar C13S
⁴available from Sigma Aldrich under catalog number D4876
⁵available from The Dow Chemical Company under tradename Versene ™ 220
⁶available from Croda Inc. under tradename Crothix-PA-(MH)
⁷available from Croda Inc. under tradename Incromide CMEA
⁸available from Stepan Company under tradename Amphosol CA
⁹preservative available from The Dow Chemical Company under tradename Neolone ™ PE
¹⁰available from The Dow Chemical Company under tradename Dowsil ™ 1785 emulsion

TABLE 3

| | CF4 | F5 | F6 | F7 |
|---|---|---|---|---|
| Ingredient INCI name | wt % | wt % | wt % | wt % |
| Deionized water | q.s. 100 | q.s. 100 | q.s. 100 | q.s. 100 |
| 30 wt % aq. soln. Sodium Lauryl | 30 | 30 | 30 | 30 |
| Ether Sulfate¹ | | | | |
| Guar hydroxypropyltrimonium | 0.3 | — | — | — |
| chloride² | | | | |
| Example S1 | — | 0.3 | — | — |
| Example S2 | — | — | 0.3 | — |
| Example S3 | — | — | — | 0.3 |
| Tetrasodium EDTA³ | 0.2 | 0.2 | 0.2 | 0.2 |
| 45 wt % aq. soln. PEG-150 | 1.0 | 1.0 | 1.0 | 1.0 |
| Pentaerythrityl Tetrastearate⁴ | | | | |
| Cocamide MEA⁵ | 1.0 | 1.0 | 1.0 | 1.0 |
| 30 wt % aq. soln. Cocamidopropyl | 6.0 | 6.0 | 6.0 | 6.0 |
| Betaine⁶ | | | | |
| Phenoxyethanol and | 0.5 | 0.5 | 0.5 | 0.5 |
| Methylisothiazolinone⁷ | | | | |
| 50 wt % aq. soln. Dimethiconol and | 2.0 | 2.0 | 2.0 | 2.0 |
| TEA-dodecylbenzenesulfonate⁸ | | | | |

¹available from Stepan Company under tradename Steol ® CS-330
²available from Solvay Novecare under tradename Jaguar C13S
³available from The Dow Chemical Company under tradename Versene ™ 220
⁴available from Croda Inc. under tradename Crothix-PA-(MH)
⁵available from Croda Inc. under tradename Incromide CMEA
⁶available from Stepan Company under tradename Amphosol CA
⁷preservative available from The Dow Chemical Company under tradename Neolone ™ PE
⁸available from The Dow Chemical Company under tradename Dowsil ™ 1785 emulsion

The shampoo formulations was prepared in each of Comparative Examples CF1-CF4 and Examples F1-F7 using the following process: In a container, the 30 wt % aqueous solution of sodium lauryl sulfate was dissolved in deionized water and heated to 70° C. with constant stirring. The polymer noted in TABLES 2 and 3 was then added to the container with stirring (e.g., polyquaternium-10, guar hydroxypropyltrimonium chloride, unmodified dextran or cationic dextran prepared according to Example S1). When the polymer was dissolved, the tetrasodium EDTA was then added to the container. Once the container contents reached 70° C., the 45 wt % aqueous solution of PEG-150 pentaerythrityl tetrastearate and the 30 wt % aqueous solution of cocamide MEA were added to the container. Then the 30 wt % solution of cocamidopropyl betaine was added to the container. The contents of the container were then allowed to cool. Once at room temperature, the phenoxyethanol and methylisothiazolinone preservative and the 50 wt % solids aqueous emulsion of dimethiconol and TEA-dodecylbenzenesulfonate were added to the container. The final pH of the product shampoo formulation was then adjusted to a pH of 5.5 using citric acid as necessary and sufficient water was added to adjust the total formulation weight to 100 g.

### Silicone Deposition Analysis

The silicone deposition on hair from the shampoo formulations prepared according to Comparative Examples CF1-CF4 and Example F1-F7 was quantified using X-ray photoelectron spectroscopy (XPS), which gives a quantitative elemental and chemical state information from the top 10 nm of the hair sample.

Hair tresses (2 g, European Virgin Brown available from International Hair Importers) were initially washed in a 9 wt % sodium laureth sulfate (SLES) solution and rinsed with water flowing at 0.4 L/min for 30 seconds. Following the initial wash step, the hair tresses were then washed with a shampoo formulation of Comparative Examples CF1-CF4 and Example F1-F7 by applying 0.8 g of the shampoo formulation to the hair tress and massaging in for 30 seconds on each side and then rinsing with water flowing at 0.4 L/min for 15 seconds on each side. The hair tresses were then evaluated using XPS. The XPS data were acquired from four areas per tress across 1 cm². The instrument parameters used are provided in TABLE 4. The mol % of the silicon from the shampoo formulation deposited on the hair is reported in TABLE 5.

TABLE 4

| | |
|---|---|
| Instrument | K-Alpha X-ray photoelectron |
| | spectroscopy (XPS) |
| X-ray source: | Monochromatic Aluminum K-alpha |
| Analyzer Pass | 200 eV (survey spectra) |
| Energy | 20 eV (high resolution spectra) |
| Take-Off Angle | 90° |
| Auto height | on |
| Analysis Area | 400 μm oval |
| Flood Gun | on |

TABLE 5

| | Kjeldahl nitrogen | Si deposition |
|---|---|---|
| Shampoo Formulation | (wt %) | (mol %) |
| Comparative Example CF1 | 0.95 | 0.7 |
| Comparative Example CF2 | 0 | 0.7 |
| Comparative Example CF3 | 1.45 | 7.2 |
| Comparative Example CF4 | 1.45 | 2.0 |
| Example F1 | 1.41 | 18.1 |
| Example F2 | 1.30 | 1.0 |
| Example F3 | 1.19 | 1.1 |
| Example F4 | 1.32 | 18.3 |
| Example F5 | 1.41 | 16.3 |
| Example F6 | 1.30 | 3.1 |
| Example F7 | 1.19 | 3.5 |

We claim:

1. A hair care formulation, comprising:
a dermatologically acceptable vehicle;
a dermatologically acceptable silicone; and
a deposition aid polymer, wherein the deposition aid polymer is a cationic dextran polymer, comprising a dextran polymer functionalized with quaternary ammonium groups;
wherein the dextran polymer has a weight average molecular weight of 100,000 to 650,000 Daltons; and wherein the deposition aid polymer enhances deposition of silicone from the cleansing formulation onto mammalian hair.

2. The hair care formulation of claim 1, wherein the hair care formulation is selected from the group consisting of a shampoo, a conditioning shampoo, a leave on hair conditioner and a rinse off hair conditioner.

3. The hair care formulation of claim 2, wherein the hair care formulation is selected from the group consisting of a shampoo and a conditioning shampoo.

4. The hair care formulation of claim 3, wherein the cationic dextran polymer has a Kjeldahl nitrogen content corrected for ash and volatiles, TKN, of 1.0 to 4.0 wt %.

5. The hair care formulation of claim 4, wherein the dextran polymer is a branched chain dextran polymer.

6. The hair care formulation of claim 5, further comprising a hair care cleansing surfactant.

7. The hair care formulation of claim 6, further comprising a chelating agent and a thickener.

8. The hair care formulation of claim 7, wherein the hair care cleansing surfactant comprises a mixture of sodium lauryl ether sulfate, cocamide monoethanolamine and cocamidopropyl betaine; wherein the chelating agent comprises tetrasodium ethylenediaminetetraacetic acid; and wherein the thickener comprises PEG-150 pentaerythrityl tetrastearate.

9. The hair care formulation of claim 8, further comprising a preservative.

10. A method of depositing silicone on to mammalian hair, comprising:
selecting a hair care formulation according to claim 1;
applying the hair care formulation to mammalian hair.
